(19) United States
(12) Patent Application Publication
(10) Pub. No.: US 2003/0220351 A1
(43) Pub. Date: Nov. 27, 2003

(54) Enteric coated caffeine tablet

(76) Inventors: Gilbert Gonzales, New York, NY (US); Keith A. Moore, Loveland, OH (US); James L. Young, West Chester, OH (US)

Correspondence Address: Timothy N. Thomas, Woodard, Emhardt, Naughton, Moriarty and McNett, Bank One Center/Tower, 111 Monument Circle, Suite 3700, Indianapolis, IN, 46204-5137, US

(21) Appl. No.: 10/154,629

(22) Filed: May 24, 2002

(51) Int. Cl. A61K 31/522; A61K 9/24; A61K 9/36

(52) U.S. Cl. 514/263.33; 424/471

(57) ABSTRACT

An enteric-coated caffeine delivery system includes a caffeine-containing core and an enteric coating made of methacrylic acid copolymer. The caffeine delivery system may also include a subcoating. The caffeine delivery system resists disintegration and release of the caffeine at a pH less than 5, but disintegrates rapidly to release the caffeine at a pH greater than about 6.

### FIELD OF THE INVENTION

The present invention relates generally to caffeine tablets, and more particularly to an enteric coated caffeine tablet. The invention allows an orally ingested, therapeutically effective amount of solid-form caffeine to pass through a patient's stomach intact before absorption in the intestine, thereby avoiding irritation and complications in the upper gastrointestinal tract.

### BACKGROUND OF THE INVENTION

Cancer patients and patients with terminal illnesses are often treated with narcotic analgesics (opioids) to counteract pain associated with the illnesses. As patents live longer with their disease, the pain they experience frequently worsens requiring escalating doses of opioids to maintain a desired level of relief. One of the most common side effects associated with the use of opioids is sedation of the patient.

Opioids are particularly sedating when first given to patients. The sedation effect tends to diminish with chronic, non-escalating use of opioids due to the phenomenon of physical tolerance. Over time, however, the dosage must often be increased to maintain the same level of pain management leading again to sedation. This escalating opioid dosage is the main reason a significant number of pain management patients require stimulant therapy.

One common stimulant frequently used to counteract opioid induced sedation is caffeine. Typically, over-the-counter, solid-dose formulations, as well as coffee and other caffeine-containing beverages, are used as caffeine delivery devices.

Patients treated with over-the-counter, solid-form caffeine products often initially experience relief from sedation. Over time, however, the repeated ingestion of caffeine required to counteract sedation leads to gastrointestinal complications. For example, regurgitation of stomach acids, frequent heartburn and bitterness are frequently associated with prolonged ingestion of caffeine into the stomach. These adverse effects may lead patients being treated with caffeine to cease using the caffeine resulting in increased sedation. They may also affect people using solid caffeine formulations as a “stimulant” or “alertness aid.”

A need therefore exists for caffeine formulations that avoid the problems associated with chronic caffeine consumption. The present invention addresses that need.

### SUMMARY OF INVENTION

Briefly describing one aspect of the present invention, there is provided an enteric-coated pharmaceutical composition having caffeine as the active ingredient. The coated composition is formulated to pass through the stomach without dissolving, and then to dissolve and release its dose of caffeine in the small and/or large intestine.

Specific objects, embodiments, forms, benefits, aspects, features and advantages of the present invention are identified in the description, examples, and claims provided herein.

### DESCRIPTION OF THE PREFERRED EMBODIMENT

For the purposes of promoting understanding of the principles of the invention, reference will now be made to the preferred embodiments. It will nevertheless be understood that no limitation of the scope of the invention is hereby intended, with alterations, modifications, and further applications of the principles of the invention described herein being contemplated as would normally occur to one skilled in the art.

As briefly described above, the caffeine-containing composition of the present invention is formulated to pass through the stomach without dissolving, yet is formulated to dissolve and release its dose of caffeine after reaching the intestine. To accomplish that end, the inventive pharmaceutical composition preferably comprises a caffeine-containing core and an enteric coating. Binders, filler, disintegrants, and/or lubricants may also be included to aid in the manufacture and/or delivery of the formulation. In some embodiments the composition also includes a subcoating between the caffeine-containing core and the enteric coating.

The core of the composition includes an active ingredient comprising pharmaceutical-grade caffeine. Pharmaceutical-grade caffeine is commercially available, and is well known to the art. In some preferred embodiments the active ingredient consists essentially of pharmaceutical-grade caffeine.

The exact amount of caffeine depends on the intended use of the coated composition. In general though, the amount is a therapeutically-effective amount for a particular medical use. For example, the amount of caffeine necessary to be therapeutically effective for reversing the sedation effects associated with opioid pain management will vary from patient to patient, but generally ranges from 50-300 mg. Caffeine levels of 50 mg to 500 mg are preferred for other indications. The caffeine preferably comprises 40-70% of the weight of the total formulation, with caffeine comprising 50-60% of the composition in the most preferred embodiments.

The core of the inventive composition may also include one or more inactive ingredients, such as binding agents, fillers, lubricants, disintegrants, etc. Preferably, the binding agent is microcrystalline cellulose although other suitable binders may also be used. Examples of other binders include povidone, methylcellulose, hydroxypropylmethylcellulose, polyvinylpyrollidine, acacia, gelatin, and sucrose. In the preferred embodiments the binder comprises between 30% and 50% of the weight of the total composition, with the binder comprising between 35% and 40% in the more preferred embodiments.

Magnesium stearate is preferred for lubricating the compaction and tableting process of the core. Examples of other lubricants include stearic acid, talc, hydrogenated vegetable oils, and metallic stearates. In some preferred embodiments the lubricant comprises less than 1% of the weight of the total composition, with a lubricant amount of less than 0.5% being more preferred.

One or more disintegrants such as cellulose, alginic acid, sodium starch glycolate, croscarmellose sodium, modified starches, and Explotab® manufactured by the Penwest Pharmaceuticals Co. may be used to speed disintegration of the core once the enteric coating is dissolved in the intestine.

In one preferred embodiment the core is a solid mass of active (and optionally inactive) ingredients that are compressed into a tablet. In other embodiments, the “core” is a powder or combination of powders (or granules, or micro-pellets, etc.) that may be formed into a tablet or contained in a capsule, as is known to the art. All enteric-coated solid dose formulations are believed to be included within the metes and bounds of the broadest aspects of the present invention, with the exception of embodiments in which the enteric coating surrounds an active ingredient layer provided around an inert core. In the present invention, the core comprises one or more active ingredients.

When the solid tablet form is desired, the core ingredients are preferably passed through a mesh screen to remove any lumps prior to mixing. The resulting core mixture is then compressed into a tablet form using a suitable tableting press. The tablet may be a round, biconvex tablet, although other shapes may also be used.

The preferred tablets are approximately 7 mm in diameter, with the smaller size allowing the tablets to pass through the stomach more easily. The tablets preferably have a hardness of 8-12 kp, with a hardness of 9-10 kp being more preferred.

A preferred formulation for preparing an uncoated tablet core having 100 mg of caffeine is set out below.

| | Amount per |
|---|---|
| Material | Tablet (mg) |
| Tablet Core | |
| Caffeine | 100.00 |
| Microcrystalline | 75.00 |
| Cellulose | |
| Magnesium | 0.44 |
| Stearate | |
| Uncoated Tablet | 175.44 |
| Net Weight | |

The caffeine-containing core is coated with an enteric coating. As is known to the art, an enteric coating for pharmaceutical compositions is a coating that releases the active ingredient in the intestine. Accordingly, the caffeine-containing composition of the present invention is coated with a coating effective to prevent release of the active ingredient as the composition passes through the patient's stomach (which normally takes several hours at a pH of less than 5), while allowing release of the active ingredient once the composition reaches the small intestine (which normally has a pH of at least about 6).

The preferred enteric coating is made from a methacrylic acid copolymer, such as Eudragit® L100-55 available from Rohm Pharma, GMBH. Eudragit® L100-55 is an aqueous acrylic resin dispersion of an anionic copolymer comprised of methacrylic acid and ethyl acrylate. One commercially available preparation of Eudragit® L100-55 is Acryl-eze™ sold by Colorcon, Inc.

Although Eudragit® L100-55 is the preferred enteric coating polymer, other suitable enteric coatings such as Sureteric® (by Colorcon, Inc) or formulated coatings such as phythalic acids or phythalic acid esters (such as polyvinyl acetate phthalate (“PVAP”)), hydroxypropylcellulose, and carboxymethylcellulose may also be used.

Preferably, the enteric coating will comprise from 5-15% by weight of the final coated composition. More preferably, the enteric coating will comprise from 6-12% by weight of the final coated composition. Most preferably, the enteric coating will comprise from about 7% to about 8% by weight of the final coated composition.

A preferred formulation for the preparation of an enteric film coating suspension to coat uncoated tablet cores is set out below.

| | Amount per 100 g |
|---|---|
| Material | Suspension (g) |
| Coating | |
| Eudragit ® L100-55 | 16.7 |
| Purified Water | 83.3 |

The final coated composition may also contain a subcoating layer between the core and the enteric coating. Preferably this subcoating layer will be a thin aqueous base coat that generally is a mixture of one or more types of hydroxypropyl methylcellulose (HPMC) such as Opadry® II White, although other suitable subcoating compositions such as cellulose polymers or modified cellulose polymers, sugars, gums with sugars, etc., may be used. Klucel® hydroxypropylcellulose (Hercules, Inc.), various Opadry® polymer compositions (Colorcon, Inc.), and Methocel™ hydroxypropyl methylcellulose polymer (Dow Chemical Co.) are examples of some commercially available subcoating compositions.

Addition of the subcoating to the core prior to application of the enteric coating allows for use of less enteric coating without reducing the stability of the coating in the low pH environment of the stomach. Preferably the subcoating layer will comprise 2% by weight of the final coated composition.

A preferred formulation for the preparation of a subcoating suspension to coat uncoated tablet cores is set out below.

| | Amount per 100 g |
|---|---|
| Material | Suspension (g) |
| Subcoating | |
| Opadry ® II White | 13.0 |
| Purified Water | 87.0 |

Preferred ranges of ingredients in coated tablets according to the most preferred embodiments of the present invention are set forth in the tables below.

| | Percentage |
|---|---|
| Material | by Weight |
| Core | |
| Caffeine | 50-54 |
| Microcrystalline Cellulose | 37-40 |
| Magnesium Stearate | 0.21-0.23 |
| Coating | |
| Eudragit ® L100-55 | 7-15 |
| Core | |
| Caffeine | 50-55 |
| Microcrystalline Cellulose | 37-41 |
| Magnesium Stearate | 0.21-0.23 |
| Subcoat | |
| Opadry ® II White | 1-4 |
| Coating | |
| Eudragit ® L100-55 | 5-10 |

The enteric coating and subcoating of the present invention may be applied to the core by any suitable means. A spray application using a coating pan type sprayer is used in one embodiment of the present invention. Other application methods such as using a fluid bed coating apparatus with a top spray mode may also be used. Once the subcoating (if used) and enteric coating are applied and dried, the resulting coated compositions may be sorted and packaged as desired.

Depending on their size, the coated compositions may be ingested individually (in the case of tablets), filled into dissolvable capsules (in the case of granules) or dispersed into a suspension in a suitable medium (in the case of micro-pellets).

The following example further describes the materials and methods used in the preferred aspects of the present invention, and is intended for illustrative purposes only. All mesh sizes are given in U.S. standard ASTM.

### EXAMPLE 1

A formulation for tablets containing 100 mg of caffeine having the following composition was prepared as described below.

| | Weight Percentage | Weight Percentage |
|---|---|---|
| COMPOSITION | of Component | of Final Formulation |
| Tablet Core | | |
| Caffeine | 57.0 | 52.059 |
| Microcrystalline Cellulose | 42.7 | 39.044 |
| Magnesium Stearate | 0.2 | 0.229 |
| Coating and Subcoat | | |
| Acryl-eze ™ | 79.0 | 6.846 |
| (dry basis) | | |
| Opadry ® II White | 21.0 | 1.822 |
| (dry basis) | | |

The preparation of caffeine tablets was begun by screening anhydrous caffeine and microcrystalline cellulose through a #20 mesh screen to remove any lumps. The initial ingredients were then added to a tumble-type blender and mixed for approximately 25 minutes. Magnesium stearate was then screened through a #30 mesh screen and added to the blender with the initial ingredients. The mixture was then blended for an additional 5 minutes.

The blended mixture was removed from the blender and weighed. The mixture was then transferred to an automatic tableting press. The press was adjusted to produce tablet cores of approximately 175.4 mg in weight and having a hardness of 10 kp. The resulting tablet cores were then placed in a storage container prior to coating.

The coating was prepared prior to application to the tablet cores in two steps. First, a subcoating was prepared by dissolving 7.0 kg of Opadry® II White in approximately 46.7 l of purified water to form a solution. Next, the enteric coating was prepared by mixing 26.3 kg of Acryl-eze™ with 131.5 l of purified water to form a dispersion.

The tablet core coating procedure utilized a coating-pan type sprayer. Prior to coating, the tablets were placed in the spray pan of the spray unit and preheated to a temperature between 50-56° C. The dual spray guns of the spray unit were primed with the subcoating solution and the spray rate adjusted. After application of the subcoating, the spray guns were primed with the Acryl-eze™ dispersion and the flow rate adjusted. A 7% weight gain due to the enteric film coating was determined to be sufficient.

After coating, the tablets were dried in the spray pan by tumbling slowing for 10-15 minutes and then removed. The resulting enteric coated caffeine tablets were found to be stable in a 0.1N solution of hydrochloric acid but dissolved readily at a pH of 7.0.

While the invention has been illustrated and described in detail in the foregoing description, the same is to be considered as illustrative and not restrictive in character, it being understood that only the preferred embodiment has been shown and described and that all changes, modifications and equivalents that come within the spirit of the inventions disclosed are desired to be protected.

What is claimed is:

1. A method of orally administering caffeine while avoiding complications in the upper gastrointestinal tract, said method comprising orally ingesting a solid-dose pharmaceutical composition comprising:
(a) a central core portion including at least one active ingredient, wherein said at least one active ingredient comprises caffeine; and
(b) an enteric coating covering said core to ensure that the caffeine is not released into the patient's stomach, but instead is released into the patient's intestine.

2. The method of claim 1 wherein said enteric coating comprises an aqueous dispersion of anionic copolymer based on methacrylic acid and ethyl acrylate.

3. The method of claim 2 wherein said enteric coating comprises Eudragit® L100-55.

4. The method of claim 1 wherein said caffeine is separated from said enteric coating by a subcoat layer.

5. The method of claim 4 wherein said subcoat layer comprises a mixture of one or more types of hydroxypropyl methylcellulose.

6. The method of claim 1 wherein said enteric coating further protects a binding agent and a lubricant to ensure that said binding agent and said lubricant are not released into the patient's stomach, but instead are released into the patient's intestine.

7. The method of claim 1 wherein said dose of caffeine comprises between 50 mg and 300 mg of caffeine.

8. The method of claim 1 wherein said central core comprises a caffeine-containing tablet.

9. The method of claim 1 wherein said central core comprises granules of caffeine.

10. The method of claim 1 wherein said central core comprises a micro-pellet of caffeine.

11. A solid-dose pharmaceutical composition, comprising:
(a) a core portion including at least one active ingredient, wherein said at least one active ingredient comprises caffeine; and
(b) an enteric coating covering said core.

12. A composition according to claim 11 wherein said enteric coating comprises an aqueous dispersion of anionic copolymer based on methacrylic acid and ethyl acrylate.

13. A composition according to claim 12 wherein said enteric coating comprises Eudragit® L100-55.

14. A composition according to claim 11 wherein said coating layer is present in said caffeine delivery system in an amount from about 5% to about 15% by weight.

15. A composition according to claim 11 wherein said core further comprises a binding agent and a lubricant.

16. A composition according to claim 11 wherein said at least one active ingredient consists essentially of caffeine.

17. A composition according to claim 11 wherein said at least one active ingredient comprises caffeine and at least one member selected from the group consisting of analgesics, anticonstipatories, antacids, and anti-secretories.

18. A composition according to claim 11, and further including a subcoat layer between said core and said coating.

19. A composition according to claim 18 wherein said subcoat is present in said caffeine delivery system in an amount of about 2% by weight.

20. A composition according to claim 11 wherein said central core comprises a caffeine-containing tablet.

21. A composition according to claim 11 wherein said central core comprises granules of caffeine.

22. A composition according to claim 11 wherein said central core comprises a micro-pellet of caffeine.

23. A pharmaceutical composition comprising:
(a) a core comprising 40-70% caffeine, 30-60% microcrystalline cellulose, and 0-2% magnesium stearate; and
(b) an enteric coating comprising a methacrylic copolymer.

24. The composition of claim 23 wherein said core comprises about 57% caffeine, about 43% microcrystalline cellulose, and about 0.2% magnesium stearate.

25. The composition of claim 23 wherein said composition further includes a subcoat layer between said core and said enteric coating.

26. The composition of claim 23 wherein said coating is present in said tablet in an amount from between about 5% to about 15% by weight.

27. The composition of claim 23 wherein said methacrylic copolymer comprises an aqueous dispersion of anionic copolymer based on methacrylic acid and ethyl acrylate.
